(19) United States
(12) Patent Application Publication
(10) Pub. No.: US 2023/0346699 A1
(43) Pub. Date: Nov. 2, 2023

(54) ADMINISTRATION OF R-BETA-HYDROXYBUTYRATE AND RELATED COMPOUNDS IN VETERINARY APPLICATIONS

(71) Applicant: AXCESS GLOBAL SCIENCES, LLC, Salt Lake City, UT (US)

(72) Inventors: Ryan P. LOWERY, Tampa, FL (US); Jacob WILSON, Tampa, FL (US)

(21) Appl. No.: 18/140,493

(22) Filed: Apr. 27, 2023

Related U.S. Application Data
(60) Provisional application No. 63/336,267, filed on Apr. 28, 2022.

(51) Int. Cl. A61K 9/00; A61K 31/12; A61K 31/198; A61K 31/4172; A61K 31/202; A61K 31/4745; A61K 31/19; A23K 20/105; A23K 20/142; A23K 20/158; A23K 20/137; A23K 50/40; A23K 50/20; A23K 50/10; A23K 50/30

(52) U.S. Cl. CPC A61K 9/0095; A61K 31/12; A61K 9/0056; A61K 31/198; A61K 31/4172; A61K 31/202; A61K 31/4745; A61K 31/19; A23K 20/105; A23K 20/142; A23K 20/158; A23K 20/137; A23K 50/40; A23K 50/20; A23K 50/10; A23K 50/30

(57) ABSTRACT

A ketone body composition comprising beta-hydroxybutyrate, related compounds (e.g., 1,3-butanediol), and/or one or more other compounds (e.g., butyrate) may be administered to a non-human animal to raise blood ketones and improve animal health. For example, the ketone body composition can be administered to non-human mammal in order to therapeutically or prophylactically treat one or more conditions selected from obesity, muscle atrophy, body composition, gut health, disease, cancer, epilepsy, seizures, Alzheimer's, oxidative stress, vascular disease, disrupted mitochondria, metabolic decline, cognitive decline, brain function, brain metabolism, brain atrophy, mental acuity, neurological disorders, inflammation, joint health, motor control, memory, or mood. The ketone body composition can be encapsulated, incorporated into a microemulsion or into liposomes, agglomerated, processed using masking/flavoring technologies, and/or otherwise processed so as to improve taste and/or reduce organoleptic reactions from the non-human mammal. Example non-human animals include dogs, cats, horses, cattle, sheep, and pigs.

### CROSS -REFERENCE TO RELATED APPLICATIONS

This application claims benefit of U.S. Provisional Application No. 63/336,267, filed Apr. 28, 2022, which is incorporated herein by reference in its entirety.

### TECHNICAL FIELD

The present invention relates to administration of butyrate, beta-hydroxybutyrate, and related compounds in non-human animals.

### BACKGROUND

While ketogenic diets have been popular in humans, human-styled ketogenic diets alone have proved ineffective in dogs. Blood ketone levels are only modestly elevated in dogs after ketogenic diets and fasting, reaching only a fraction of the blood ketone levels usually seen in humans.

### SUMMARY

While ketogenic diets alone have lacked success in increasing blood ketone levels in non-human animals, administration of beta-hydroxybutyrate, related compounds, and optionally other compounds, may increase blood ketone levels as presently described. Thus, administration of an effective amount of beta-hydroxybutyrate in non-human mammals increases blood ketone levels and improves the health of the animal.

For example, the effective amount of the beta-hydroxybutyrate, related compounds, and/or one or more other compounds may be administered by means of a ketone body composition to therapeutically or prophylactically treat one or more conditions, including but not limited to, cause weight loss, weight maintenance, elevate blood ketone levels, maintain blood ketone levels, reduce blood glucose levels, maintain blood glucose levels, reduce or reverse disease, reduce or reverse the growth of cancerous tumors, treat epilepsy and/or symptoms thereof, treat Alzheimer's and/or symptoms thereof, treat or reduce seizures, improve brain metabolism, improve brain function and/or cognitive health, decrease cognitive decline, decrease brain atrophy, improve body composition (e.g., increase lean to fat ratio), increase fat loss, reduce inflammation, improve joint health, improve gut health, etc. The pharmaceutically effective amount of beta-hydroxybutyrate, butyrate, related compounds, and/or combinations thereof may be administered to healthy individuals and/or unhealthy individuals (e.g., with diseases and/or disorders).

In various implementations, a ketone body component such as beta-hydroxybutyrate, related compounds, and optionally other compounds, may be administered to animals such as large animals (e.g., horses, cattle (e.g., cows and bulls), sheep, pigs, etc.) and/or medium animals (e.g., dogs, cats, etc.), and/or small animals in order to therapeutically or prophylactically treat one or more conditions disclosed herein. The beta ketone body component such as hydroxybutyrate, related compounds, and optionally other compounds (e.g., butyrate), may be administered to non-mammals, in various implementations.

The details of one or more implementations are set forth in the description below. Other features, objects, and advantages of the implementations will be apparent from the description.

### DETAILED DESCRIPTION

In various implementations, a ketone body composition comprising one or more ketone body components such as beta-hydroxybutyrate, acetoacetate, and related compounds (e.g., derivatives, esters, polymers, precursors, etc.), can be administered to non-human mammals alone or in combination with one or more other compounds (e.g., butyrate) to increase blood ketone levels and therapeutically or prophylactically treat one or more conditions disclosed herein. For example, administration of an effective amount of these compound(s) may therapeutically or prophylactically treat obesity, such as to promote and/or maintain weight loss.

In some implementations, blood ketone levels and/or blood glucose levels may be increased and/or maintained within a predetermined range when an effective amount of one or more ketone body compounds or precursors are administered. In some implementations, health of a non-human mammal (e.g., body composition, brain health, cognition, disease, strength, symptoms of disease, mental acuity, fasting glucose levels, inflammation, joint health etc.) may be improved and/or maintained by administration of the ketone body component that includes beta-hydroxybutyrate and/or related compounds (e.g., derivatives, esters, polymers, butyrate, etc.).

While ketone bodies such as beta-hydroxybutyrate and related compounds have been administered in humans, non-human mammals and humans have different metabolisms and different responses to similar treatments. Unlike in humans, ketogenic diets and/or fasting in animals are not associated with elevated blood ketone levels that produce a beneficial impact on health. Thus, the ability of the ketone body component, such as beta-hydroxybutyrate (e.g., R-beta-hydroxybutyrate) and related compounds, to elevate blood ketone levels and therapeutically or prophylactically treat one or more conditions disclosed herein produce beneficial impact(s) on health is unexpected and of greater importance in animal health.

Aging is associated with higher rates of inflammation, reduced muscle, higher body fat percentage, joint disease, cancer, and other diseases. An additional hallmark of aging is a decline in brain function, resulting in reduced cognition and/or behavioral changes. Age-associated declines in brain function include atrophy of neurons, white, and gray matter, and abnormal metabolism, including greater inflammation, oxidative stress, vascular disease, and disrupted mitochondria and energy metabolism. Ultimately, these structural declines can result in severe disease, including epilepsy, seizures, and Alzheimer's. Since animals, unlike humans, have difficulty expressing their symptoms to human caretakers (e.g., early symptoms and progression of symptoms) that might indicate these age-related issues, preventative treatment and symptom reduction treatment, such as the benefits associated with administration of beta-hydroxybutyrate, are critical.

The brain is a very metabolically active organ, accounting for roughly 25% of the body's total resting energy metabolism (Cunnane, et al., 2011). The brain is primarily reliant on glucose as an energy source, and is particularly vulnerable and intolerant to disruptions in energy metabolism. With age, glucose metabolism progressively declines, leading to abnormal metabolism. Ketone bodies, including acetoacetate and B-hydroxybutyrate, are important alternative sources of energy for the brain. Interestingly, brain ketone metabolism appears to be unaffected by age, and can be oxidized at a rate seven to nine times faster than glucose, providing up to 70% of the brain's energy during prolonged periods of fasting (May et al., 2019). Accordingly, nutritional methods that elevate blood ketones have been of interest as a therapy target.

Numerous studies have successfully used ketogenic diets in weight management, and they have potential anti-inflammation properties. Ketogenic diets have also been used effectively for refractory epilepsy in humans. Further, there is rapidly accumulating evidence supporting the benefit of ketogenic diets for multiple other neurological disorders, including Alzheimer disease (McDonald et al., 2018). The use of ketogenic diets in other species, including dogs, is also of interest. However, dogs differ from humans.

In dogs, overall brain metabolism can decline as much as 25% by six years of age and is accompanied by progressive impairments in cognition (London et al., 1983). Early interventions are an important opportunity to slow the progression of brain disease, thereby supporting the human-animal bond, well-being, and quality of life. Among cognitive diseases in dogs, seizures and epilepsy are the most common neurological signs affecting an estimated 0.6%-0.8% of the population (Erlen et al., 2018). Antiseizure drugs (ASDs) can be effective, but also have undesirable drug-related adverse effects. Further, approximately a third of dogs are unresponsive to drug therapy and continue having seizures (Berk et al., 2020). Therefore, new and complementary treatments are important for dogs.

Nutritional methods to reduce or eliminate seizures is of immense interest, with research showing that 60% of dog owners changed their dog's diet after the dog was diagnosed with idiopathic epilepsy (Berk et al., 2020). Ketogenic diets have been one such dietary intervention used. However, unlike in humans, ketogenic diets have proved ineffective in dogs, resulting in no improvement in seizure frequency versus control diets. Further, blood ketone levels are only modestly elevated in dogs after ketogenic diets and fasting, reaching only a fraction of blood ketone levels usually seen in humans. De Bruijne and Van den Brom (1986) established that dogs have a higher rate of clearance of plasma ketones than humans. Thus, the seemingly low concentration of beta-hydroxybutyrate in dogs is not from reduced production of ketones, but rather, from different metabolisms compared to humans, resulting in higher rates of peripheral utilization in dogs.

The concentration of beta-hydroxybutyrate in the brain and cerebral spinal fluid (CSF) is directly proportional to the concentration found in plasma, and it increases as the duration of fasting continues. When available, beta-hydroxybutyrate is preferentially utilized over glucose, lactate, and pyruvate by neurons as an energy substrate. So even a modest increase in plasma concentrations of ketones could still provide a significant contribution of energy for the brain and neurons. Thus, interventions that can more effectively raise blood ketones may prove effective in dogs.

In various implementations, ketone body administration in dogs as a therapy may improve body composition, aid in fat loss, reduce inflammation, improve and/or maintain joint health, and/or improve and/or maintain cognitive health (e.g., including treatment for seizures and Alzheimer's symptoms). Ketone bodies, such as R-beta-hydroxybutyrate, may be effective alone, or in combination with fatty acids, such as omega-3, omega-6, and omega-9 fatty acids, ketogenic diets, and/or antiseizure medications. In some implementations, fatty acids may include even and/or odd fatty acids in the range of six to 12 carbons, free form fatty acids, natural form fatty acids derived from coconut oil, dairy, and/or palm kernel oil.

The ketones administered may include beta-hydroxybutyrate (e.g., racemic and/or nonracemic) and/or derivatives of beta-hydroxybutyrate. In some implementations, the composition may include fatty acids, other ketones and/or combinations thereof. In some implementations, the composition may include approximately 1 to approximately 20 grams of fatty acids. In some implementations, approximately 1 to approximately 15 grams of BHB may be delivered orally. The composition may be administered one to four times daily. The administration of the composition may aid in fat loss, reduce inflammation, improve or maintain joint health, reduce the incidence of and/or symptoms of seizures, reduce the incidence of and/or symptoms of Alzheimer's, and/or reduce the incidence of and/or symptoms of cognitive decline in non-human mammals, such as dogs.

In some implementations, medium animals such as dogs and cats may be administered a composition that includes approximately 0.5 g to approximately 20 g of beta-hydroxybutyrate. In some implementations, large animals such as cattle (e.g., cows and bulls) and horses may be administered a composition that includes approximately 20 g to approximately 200 g of beta-hydroxybutyrate, such as approximately 20 g to approximately 100 g of beta-hydroxybutyrate, or approximately 50 g to approximately 200 g of beta-hydroxybutyrate, preferably approximately 50 g to approximately 100 g of beta-hydroxybutyrate, depending on the size and/or age of the animal.

Additional compositions as described herein may be administered with the beta-hydroxybutyrate, including but not limited to amino acid(s), protein(s), vitamin(s), mineral(s), herb(s), extracts of herb(s) (e.g., AC-11 and/or CMED 100), CBD (cannabidiol), fatty acids or esters thereof, triglycerides, pharmaceutically acceptable binders, and/or carriers, etc. In some implementations, the composition may be infused with or coated onto a portion of an animal treat (e.g., bone, lick, etc.). In some implementations, the composition may be included in supplements and/or food (e.g., bone broth packets, etc.). In some implementations, the delivery method may provide additional benefits, such as a composition that includes beta-hydroxybutyrate and bone broth to promote joint health. As another example, a composition that includes beta-hydroxybutyrate and amino acids to reduce the symptoms associated with aging by improving muscle health and/or mass.

In various implementations, administration of the composition may induce and/or maintain ketosis. Administration of the composition may induce and/or maintain weight loss. Administration of the composition may increase fat loss and/or improve body composition (e.g., increase muscle to fat ratio). Administration of the composition may improve brain function (e.g., memory, mood, cognitive function, motor control). Administration of the composition may reduce inflammation and/or guard against some inflammation. Administration of the composition may improve physical performance (e.g., speed, dexterity, etc.). Administration of the composition may reduce cognitive decline and/or reduce symptoms associated therewith (e.g., energy, focus, mood, motor control, etc.). Administration of the composition may slow or reverse the growth of cancerous tissues. Administration of the composition may increase blood ketone levels.

In various implementations butyrate, beta-hydroxybutyrate, and/or related compounds may be administered to non-human mammals. Beta-hydroxybutyrate (e.g., R-beta-hydroxybutyrate, L-beta-hydroxybutyrate, and/or D,L-beta-hydroxybutyrate) may include beta-hydroxybutyrate salts and/or beta-hydroxybutyrate esters. In some implementations, beta-hydroxybutyrate may include beta-hydroxybutyrate bound to another compound (e.g., one or more amino acids) and/or polymers or oligomers of beta-hydroxybutyrate.

For example, beta-hydroxybutyrate (e.g., R-beta-hydroxybutyrate, L-beta-hydroxybutyrate, and/or D,L-beta-hydroxybutyrate) may include one or more beta-hydroxybutyrate salts, such as beta-hydroxybutyrate sodium salt (e.g., sodium beta-hydroxybutyrate), beta-hydroxy butyrate potassium salt (e.g., potassium beta-hydroxybutyrate), beta-hydroxybutyrate calcium salt (e.g., calcium beta-hydroxybutyrate), beta-hydroxybutyrate magnesium salt (e.g., magnesium beta-hydroxybutyrate), beta-hydroxybutyrate lithium salt (e.g., lithium beta-hydroxybutyrate), sodium beta-hydroxybutyrate, arginine beta-hydroxybutyrate, lysine beta-hydroxybutyrate, histidine beta-hydroxybutyrate, ornithine beta-hydroxybutyrate, creatine beta-hydroxybutyrate, agmatine beta-hydroxybutyrate, or citrulline beta-hydroxybutyrate, other appropriate organic salts that include beta-hydroxybutyrate, and/or combinations thereof. In some implementations, the beta-hydroxybutyrate may include mixed beta-hydroxybutyrate salts including calcium, sodium, magnesium, potassium, zinc, selenium, chromium, other appropriate minerals, and/or combinations thereof.

In some implementations, the beta-hydroxybutyrate may be complexed and/or coupled to another compound (e.g., amino acid and/or berberine) and a beta-hydroxybutyrate salt may include a complex (e.g., chelate) that includes a mineral (e.g., calcium, zinc, etc.) and the beta-hydroxybutyrate compound coupled to another compound. The beta-hydroxybutyrate may include single isomer beta-hydroxybutyrate and/or polymer beta-hydroxybutyrate. For example, R-beta-hydroxybutyrate may include single isomer R-beta-hydroxybutyrate and/or polymer R-beta-hydroxybutyrate.

In some implementations, beta-hydroxybutyrate may be administered along with 1,3-butanediol, medium chain fatty acid or triglyceride (MCT), or a ketone body ester, such as ethyl acetoacetate and/or ethyl beta-hydroxybutyrate.

Beta-hydroxybutyrate may be in the form of a racemic mixture and/or include individual isomers of beta-hydroxybutyrate. In some implementations, one or more specific chiralities of beta-hydroxybutyrate may be utilized. For example, R-beta-hydroxybutyrate (also referred to as D-beta-hydroxybutyrate), S-beta-hydroxybutyrate (also referred to as L-beta-hydroxybutyrate), and/or mixtures (e.g., racemic mixtures) thereof may be utilized. In some implementations, R-beta-hydroxybutyrate may be included in the composition (e.g., a more purified form of R-beta-hydroxybutyrate rather than D,L-beta-hydroxybutyrate). For example, enriched R-beta-hydroxybutyrate may include less than approximately 10 percent, less than approximately 5 percent, or less than approximately 1 percent L-beta-hydroxybutyrate.

Enriched or optically pure R-beta-hydroxybutyrate may have a greater bioavailability than other chiralities of beta-hydroxybutyrate. R-beta-hydroxybutyrate may have a greater impact on the health of an individual (e.g., due to decreased side effects; increase ketone levels, weight loss, mental acuity, fat loss, etc.) than L-beta-hydroxybutyrate and/or D,L-beta-hydroxybutyrate. In some implementations, R-beta-hydroxybutyrate may result in improvements in health not capable by L-beta-hydroxybutyrate and/or D,L-beta-hydroxybutyrate. R-beta-hydroxybutyrate may have less impurities due to manufacturing, such as less crotonic acid (e.g., which can be harmful to individuals), than other forms of beta-hydroxybutyrate (e.g., L-beta-hydroxybutyrate and/or D,L-beta-hydroxybutyrate). In some implementations, R-beta-hydroxybutyrate may be better able to bind with other compounds (e.g., purine, lysine, potassium, and/or other amino acids; dihydroberberine; etc.) to deliver the beta-hydroxybutyrate to a non-human mammal. Thus, R-beta-hydroxybutyrate (e.g., greater than 90 percent purity of R-beta-hydroxybutyrate and less than 10 percent L-beta-hydroxybutyrate) and/or mixtures with R-beta-hydroxybutyrate may be administered to non-human mammals.

In some implementations, unexpectedly, a smaller amount of R-beta-hydroxybutyrate may be as pharmaceutically effective (e.g., in increasing and/or maintaining weight loss; in increasing and/or maintaining elevated ketone levels, etc.) or more pharmaceutically effective as D,L-beta-hydroxybutyrate (e.g., racemic mixture of D- and L-beta-hydroxybutyrate). For example, approximately half an amount of R-beta-hydroxybutyrate may be administered to achieve approximately the same efficacy as D,L-beta-hydroxybutyrate and/or L-beta-hydroxybutyrate. The R-beta-hydroxybutyrate may be more bioavailable than other chiralities of beta-hydroxybutyrate and thus allow a smaller effective amount than other chiralities. Thus, by utilizing R-beta-hydroxybutyrate, the administration amount of beta-hydroxybutyrate can be reduced (e.g., when compared to the administration amount of D,L-beta-hydroxybutyrate) while providing an effective amount, such as, e.g., for weight loss and/or maintenance; for elevating and/or maintaining blood ketone levels, etc. Reducing the amount of beta-hydroxybutyrate, when the beta-hydroxybutyrate is provided in salt form, may reduce a user's intake of the cation of the salt (e.g., sodium, potassium, etc.). Since intake of some of these cations in beta-hydroxybutyrate salts, such as sodium, potassium, magnesium, and calcium, in amounts greater than a predetermined recommended amount may cause health problems (e.g., organ damage, gastrointestinal problems, etc.), reducing the amount of beta-hydroxybutyrate salt by using enriched or optically pure R-beta-hydroxybutyrate may inhibit side effects and/or health problems associated salts combined with beta-hydroxybutyrate administration in users.

Administration of beta-hydroxybutyrate may cause weight loss and/or weight maintenance; elevated beta-hydroxybutyrate levels in the blood; elevated, reduced, and/or maintenance of blood ketone levels; induction and/or maintenance of ketosis; improve mental acuity; improve focus; improve energy; improve cognitive function; reduce traumatic brain injury; improve glucose tolerance; decrease blood glucose levels; reduce neurological disorders and/or symptoms thereof; improve cancer and/or symptoms thereof; improve inflammatory conditions; suppressing appetite; improve symptoms associated with aging; provide anti-glycation affects; improve epilepsy and/or symptoms thereof; improve mood; improve performance; improve strength; increase muscle mass; increase fat loss; improve body composition; improve energy; improve focus; improve cognitive function; improve gut functions; and/or combinations thereof.

The beta-hydroxybutyrate (e.g., R-beta-hydroxybutyrate) may be administered in healthy and unhealthy non-human mammals (e.g., non-human mammals with diseases and/or disorders).

In some implementations, the beta-hydroxybutyrate, such as R-beta-hydroxybutyrate, may be administered with and/or coupled to a compound such as an amino acid. For example, beta-hydroxybutyrate may be coupled to (e.g., chemically bonded to) amino acids, such as leucine, lysine, arginine, histidine, ornithine, creatine, agmatine, citrulline and/or combinations thereof. In some implementations, R-beta-hydroxybutyrate may be utilized rather than other chiralities since R-beta-hydroxybutyrate may be more easily bound to leucine, purine, lysine, and/or other amino acids. Administration of beta-hydroxybutyrate that is coupled to an amino acid may reduce the intake of cations associated with beta-hydroxybutyrate salts (e.g., which may inhibit side effects associated with administration) and/or allow administration of another compound that has health benefits (e.g., administration of some amino acid may promote smooth muscle growth and/or cell repair). In some implementations, approximately 0.5 g to approximately 10 g of amino acid may be administered with a beta-hydroxybutyrate. For example, the composition may include beta-hydroxybutyrate and approximately 60 mg of an amino acid, such as leucine, and may be administered daily. In some implementations, approximately 0.5 g to approximately 2 g of an amino acid, such as leucine, may be administered with a beta-hydroxybutyrate. For example, the composition may include beta-hydroxybutyrate and approximately 1-3 g of leucine. The R-beta-hydroxybutyrate and the leucine may be a mixture, administered separately and proximate in timing, a complex, and/or administered in any other appropriate manner.

In some implementations, the composition may include a R-beta-hydroxybutyrate salt and beta-hydroxybutyrate-amino acid complex (e.g., beta-hydroxybutyrate bound to amino acid, such as R-beta-hydroxybutyrate-leucine complex). For example, an individual may be administered a first weight amount of sodium beta-hydroxybutyrate and a second weight amount of beta-hydroxybutyrate amino-acid complex. The first amount and the second amount may be different or the same.

In some implementations, the beta-hydroxybutyrate composition may include one or more beta-hydroxybutyrate salts and/or one or more beta-hydroxybutyrate esters. For example, a non-human mammal may be administered a first weight amount of sodium beta-hydroxybutyrate and a second weight amount of beta-hydroxybutyrate ester. The first amount and the second amount may be different or the same. The beta-hydroxybutyrate salt and the beta-hydroxybutyrate ester may be a bound complex, a mixture of compounds, and/or separately administered approximately concurrently.

In some implementations, the beta-hydroxybutyrate ester may be in solid, powdered form (e.g., plated beta-hydroxybutyrate ester), liquid, and/or gel form. The combination of beta-hydroxybutyrate salt and beta-hydroxybutyrate ester during administration may allow less salt to be utilized while producing a result (e.g., weight maintenance and/or loss; enhanced and/or maintained ketosis; elevated blood ketone levels; blood glucose reduction and/or maintenance; increase in energy; increase in mood; increase in performance; and/or increase in cognitive function).

In some implementations, elevated ketone levels (e.g., elevated blood ketone levels) may increase energy, mood, performance, and/or cognitive function in non-human animals. For example, the administration of the first amount of beta-hydroxybutyrate salt may cause a first level of blood ketone level, which may be maintained by processing of the second amount of the beta-hydroxybutyrate ester (e.g., as the body of the non-human mammal processes the beta-hydroxybutyrate ester the level of beta-hydroxybutyrate in the blood, and thus blood ketone level, may also increase over time to enhance and/or maintain the initial elevation caused by of the administered beta-hydroxybutyrate salt). For example, a ratio of beta-hydroxybutyrate salt to beta-hydroxybutyrate ester may be approximately 1 beta-hydroxybutyrate salt: approximately 1 beta-hydroxybutyrate ester, to approximately 1 beta-hydroxybutyrate salt: approximately 20 beta-hydroxybutyrate ester. The ratio of beta-hydroxybutyrate salt to beta-hydroxybutyrate ester may be approximately 20 beta-hydroxybutyrate salt: approximately 1 beta-hydroxybutyrate ester to approximately 1 beta-hydroxybutyrate salt: approximately 20 beta-hydroxybutyrate ester. In some implementations, a ratio of beta-hydroxybutyrate salt to beta-hydroxybutyrate ester may be approximately 1 beta-hydroxybutyrate salt: approximately 1 beta-hydroxybutyrate ester to approximately 1 beta-hydroxybutyrate salt: approximately 5 beta-hydroxybutyrate ester.

Related compounds that may be included as beta-hydroxybutyrate in the composition may include derivatives of beta-hydroxybutyrate, include esters of (R)-3-hydroxybutyrate and oligomers of (R)-3-hydroxybutyrate. Another example is beta-hydroxybutyrate esters derived from alcohols, such as altrose, arabinose, dextrose, erythrose, fructose, galactose, glucose, glycerol, gulose, idose, lactose, lyxose, mannose, ribitol, ribose, ribulose, sucrose, talose, threose, xylitol, xylose, galactosamine, glucosamine, mannosamine, N-acetylglucosamine, mannitol, sorbitol, threitol, (S)-1,2-propanediol and/or (R)-1,3-butanediol. In some implementations, a derivative of the beta-hydroxybutyrate may include structures of (R)-3-hydroxybutyric acid and an exemplary ester thereof (a glycerol monoester). The “R” enantiomer of the derivatives may be selected for inclusion in the composition in some implementations (e.g., to deliver R-beta-hydroxybutyrate with the administration of the compound).

In some implementations, butyrate, beta-hydroxybutyrate (e.g., R-beta-hydroxybutyrate), related compounds, and/or combinations thereof may be administered along with one or more additional compounds. The additional compounds may or may not be capable of independently increasing ketone levels, maintaining ketone levels, inducing ketosis, and/or maintaining ketosis. For example, additional compounds capable of independently increasing blood ketone levels may include short chain fatty acids (e.g., fatty acid with between 2 carbons and less than 6 carbons), short chain triglycerides (e.g., triglycerides with less than 6 carbons), medium chain fatty acids (e.g., fatty acid with 6-12 carbons), medium chain triglycerides (e.g., triglycerides with 6-12 carbons), long chain fatty acids (e.g., fatty acids with more than 12 carbons), long chain triglycerides (e.g., triglycerides with more than 12 carbons), and/or combinations thereof. In some implementations, short chain fatty acids and/or triglycerides may include acetate, propionate, and/or butyrate.

Medium chain fatty acids and/or triglycerides may include lauric acid and/or coconut oil, coconut milk powder, fractionated coconut oil, isolated hexanoic acid, isolated octanoic acid, isolated decanoic acid, ethoxylated triglyceride, triglyceride derivatives thereof, aldehyde triglyceride derivatives thereof, monoglyceride derivatives thereof, diglyceride derivatives thereof, triglyceride derivatives thereof, and/or alkyl esters thereof. Long chain fatty acids and/or triglycerides may include dairy products and/or palm oil.

In some implementations, a composition including R-beta-hydroxybutyrate and an additional compound that is independently capable of increasing ketone levels may increase ketone levels greater than merely the capability of each component individually (e.g., more than an additive increase). For example, the composition may include R-beta-hydroxybutyrate and an additional compound independently capable of increasing ketone levels such as caffeine. In some implementations, the composition may include beta-hydroxybutyrate and caffeine. In some implementations, the composition may include approximately beta-hydroxybutyrate as described and less than approximately 500 mg of caffeine. The composition with beta-hydroxybutyrate (e.g., R-beta-hydroxybutyrate including at least one R-beta-hydroxybutyrate salt) and caffeine may increase and or maintain ketosis, weight loss, fat loss, and/or mental acuity. In some implementations, the composition with beta-hydroxybutyrate (e.g., R-beta-hydroxybutyrate including at least one R-beta-hydroxybutyrate salt) and caffeine may increase mental processes (e.g., acuity including cognitive functioning, mood, energy, alertness, focus, performance, effects of aging, etc.); improve and/or maintain body composition; function as a therapeutic for one or more of the described conditions or disorders (e.g., treat neurological disorders); and/or combinations thereof.

In some implementations, the composition may include beta-hydroxybutyrate and an additional compound independently capable of increasing ketone levels, such as 1,3,7,9-tetramethyluric acid (commercially available as theacrine; and/or commercially available as TeaCrine® from Compound Solutions, California, USA). In some implementations, the composition may include approximately 0.5 mg to approximately 15 g of R-beta-hydroxybutyrate and less than approximately 500 mg of 1,3,7,9-tetramethyluric acid. In some implementations, the composition may include beta-hydroxybutyrate and less than approximately 500 mg of 1,3,7,9-tetramethyluric acid.

In some implementations, the composition may include cannabidiol (CBD) administered with the beta-hydroxybutyrate. As described in U.S. patent application Ser. Nos. 16/667,851 and 17/771,561 entitled “Administration of Butyrate, Beta-hydroxybutyrate, cannabidiol, and related compounds in humans,” which is incorporated herein by reference for all purposes to the extent the teachings do not conflict with the teachings herein. For example, the composition may include beta-hydroxybutyrate as described and CBD, such as approximately 5 mg to approximately 300 mg CBD.

For example, a pharmaceutically effective amount of one or more short chain fatty acids and/or one or more short chain triglycerides (e.g., butyric acid and/or butyrate) may be administered with a pharmaceutically effective amount of beta-hydroxybutyrate. In some implementations, the composition may include beta-hydroxybutyrate and approximately 0.1 g to approximately 50 g of short chain fatty acid and/or triglyceride may be administered from once a day to approximately 5 times a day. In some implementations, the composition may include beta-hydroxybutyrate and approximately 1 g of short chain fatty acid and/or triglyceride may be administered from once a day to approximately 5 times a day. In some implementations, the short chain fatty acid and/or triglyceride may include butyrate or derivatives of butyrate. Butyrate and/or derivatives of butyrate may be administered with and/or without beta-hydroxybutyrate to manage metabolic conditions, such as ketosis, and/or for other appropriate therapeutic purposes. Administered butyrate may be converted to beta-hydroxybutyrate in non-human animals, and thus may increase the amount of beta-hydroxybutyrate delivered to the user. In some implementations, administration of butyrate and beta-hydroxybutyrate may promote animal growth hormone synthesis, improve basal growth hormone secretion, increase muscle fiber cross-sectional area, inhibit intramuscular fat accumulation; reduce fat mass in a user; improve glucose metabolism; increase markers of mitochondrial biogenesis in skeletal muscle and/or whole-body oxygen consumption; reduced markers of oxidative stress and apoptosis and altered antioxidant enzyme activity; cause butyrate enhanced intracellular free cytosolic calcium levels; increase beta-hydroxybutyrate levels; and/or support barrier function(s) in the gut and/or reduce inflammation associated with ulcerative colitis. Since butyrate is processed by the body to provide beta-hydroxybutyrate, the delivery of beta-hydroxybutyrate via the butyrate may supplement the directly administered beta-hydroxybutyrate to maintain a level of beta-hydroxybutyrate in the blood (e.g., to promote ketosis, weight loss and/or management, etc.).

However, butyrate and/or butyric acid may not be palatable to non-human animals. Thus, in some implementations, butyrate and/or beta-hydroxybutyrate (e.g., R-beta-hydroxybutyrate) may be processed to reduce organoleptic reactions. For example, the butyrate and/or beta-hydroxybutyrate (e.g., R-beta-hydroxybutyrate) may be encapsulated, such as by microemulsion, liposomes, agglomeration, masking/flavoring technologies, and/or otherwise processed as appropriate to reduce organoleptic reactions from individuals administered the described composition(s). In some implementations, microencapsulated butyrate, beta-hydroxybutyrate, and/or butyric acid may be utilized (e.g., in combination with beta-hydroxybutyrate). Using microencapsulated butyrate, beta-hydroxybutyrate, and/or butyric acid (e.g., when compared with using unencapsulated forms) may increase animal satisfaction and/or compliance with an administration schedule since odor from the butyrate and/or butyric acid may be reduced and/or removed. The microencapsulated butyrate, beta-hydroxybutyrate, and/or butyric acid may be a free-flowing granular powder; dispersible in water; stable in acidic water solution for 30 minutes; allow controlled release in stomach and/or small intestines; inhibit glucose response (e.g., to any added materials); and/or allow delivery of a high butyrate content (e.g., around 70%).

In some implementations, an effective amount of butyrate may be administered via triglyceride tributyrin (e.g., glyceryl tributyrate or tributyrin), which is more palatable than butyric acid and butyrate. The butyrate via triglyceride tributyrin may be administered separately and/or in conjunction with one or more of the other described compounds (e.g., beta-hydroxybutyrate, fatty acids and/or esters, etc.). For example, up to approximately 200 mg/kg of the non-human animal may be administered (e.g., up to 3 times daily). Administration of the tributyrin may allow a delayed release of butyrate to the body as the tributyrin is processed by the body of the individual. The tributyrin may be unencapsulated and/or encapsulated (e.g., microencapsulated).

In some implementations, administration of beta-hydroxybutyrate and a short chain compound (e.g., short chain fatty acid and/or short chain triglyceride) may unexpectedly increase beta-hydroxybutyrate concentrations in the blood more than the administration of similar amounts of beta-hydroxybutyrate and medium chain compounds (e.g., short chain fatty acids and/or short chain triglycerides) and/or may increase beta-hydroxybutyrate concentrations in the blood more than each component individually.

In some implementations, a pharmaceutically effective amount of beta-hydroxybutyrate may be administered with a pharmaceutically effective amount of long chain fatty acid and/or triglyceride. For example, the composition may include beta-hydroxybutyrate and 0.1 to 50 g of long chain fatty acid may be administered to an individual between 1-5 times a day. In some implementations, the composition may include beta-hydroxybutyrate and approximately 1 g of long chain fatty acid and/or triglyceride may be administered from once a day to approximately 5 times a day.

In some implementations, beta-hydroxybutyrate, short chain compound(s) (e.g., fatty acids and/or triglycerides, butyrate), and/or medium chain compound(s) (e.g., fatty acids and/or triglycerides) may be administered approximately simultaneously and/or sequentially to an individual. For example, the composition may include beta-hydroxybutyrate, approximately 0.1 g to approximately 50 g short chain triglyceride, and approximately 0.1 g to approximately 50 g medium chain fatty acid such as lauric acid and/or coconut oil and may be administered between 1-5 times a day. In some implementations, the composition may include beta-hydroxybutyrate and approximately 1 g of short chain fatty acid and/or triglyceride and/or approximately 1 g of medium chain fatty acid and/or triglyceride may be administered from once a day to approximately 5 times a day. In some implementations, the composition may include beta-hydroxybutyrate (e.g., salts, esters, isomers, and/or other appropriate forms) and may be administered in non-human mammals. In some implementations, approximately 0.1 g to approximately 20 g butyrate may be administered in non-human mammals.

In some implementations, other compounds, such as compounds capable of independently decreasing glucose levels, may be administered with beta-hydroxybutyrate, such as berberine and/or associated metabolites (e.g., dihydroberberine and/or tetrahydroberberine). U.S. Pat. No. 10,278,961, entitled “Administration Of Dihydroberberine” to Lowery et al, describe dihydroberberine administration with ketone sensitizers in humans such as beta-hydroxybutyrate, and is hereby fully incorporated herein to the extent it does not conflict with the disclosure herein. In some implementations, one or more beta-hydroxybutyrates and/or other compounds described herein may be utilized as a ketone sensitizer with the dihydroberberine.

In some implementations, directly administering beta-hydroxybutyrate plus another compound that is processed to deliver beta-hydroxybutyrate (e.g., beta-hydroxybutyrate ester, beta-hydroxybutyrate polymer, butyrate, precursors such as 1,3-butanediol other appropriate compounds, and/or combinations thereof) over time may allow a first level of beta-hydroxybutyrate in the blood to be maintained over a period of time. For example, since the directly administered beta-hydroxybutyrate may elevate blood beta-hydroxybutyrate levels to a first concentration and this concentration may be approximately maintained over a period of time by providing additional beta-hydroxybutyrate via another compound administered approximately concurrently (e.g., 1,3,-butanediol, short chain fatty acid and/or triglyceride, beta-hydroxybutyrate ester, beta-hydroxybutyrate polymer, beta-hydroxybutyrate amino acid complex, etc.).

In some implementations, one or more other compounds may be administered (e.g., included in the composition and/or separately administered) with the butyrate (e.g., microencapsulated butyrate), beta-hydroxybutyrate (e.g., R-beta-hydroxybutyrate) and/or fatty acids or esters, such as short chain fatty acids. Other compositions may include, but are not limited to amino acids, amino acid metabolites, vitamins, minerals, coconut milk powder, flavorings, colorings, binders, electrolytes, tetrahydrobiopeterin, nucleic acids, alpha-ketoglutaric acid, alpha lipoic acid, nutritional co-factors, beta-methyl-beta-hydroxybutyrate, arginine alpha-ketoglutarate, R-alpha lipoic acid, thiamine, NAD+, NADH, riboflavin, FAD+, FADH, riboflavin-5-phosphate, niacin, nicotinic acid, niacinamide, inositol hexanicotinate, pyridoxine, pyridoxal, pyridoxamine, ascorbic acid and ascorbate salts, citric acid, malic acid, sodium benzoate, Pyridoxal-5-Phosphate, methylcobalamin, cyanocobalamin, adenosylcobalamin, hydroxycobalamin, pantothenic acid, pantetheine, potassium sorbate, acesulfame K, aspartame, sucralose, stevia, monk fruit extract, allulose, prebiotic fibers, XOS, GOS, MOS, IMO, LOS, xanthan gum and other organic gums/thickeners/suspension agents, and combinations thereof.

In various implementations, administration of a composition that includes beta-hydroxybutyrate may improve the health of an individual. R-beta-hydroxybutyrate may be capable of providing a greater impact on the health of an individual than D,L-beta-hydroxybutyrate and/or L-beta-hydroxybutyrate. Although previously unknown, L-beta-hydroxybutyrate may decrease the effectiveness of R-beta-hydroxybutyrate with respect to at least a portion of the impact on health. With respect to some impacts on health, L-beta-hydroxybutyrate may have no impact on health. In some implementations, even double the amount of D,L-beta-hydroxybutyrate may not achieve some of the same results (e.g., in health improvement) as R-beta-hydroxybutyrate. Thus, unexpectedly administration of D,L-beta-hydroxybutyrate rather than R-beta-hydroxybutyrate may not have the same impact on health and/or have less of an impact on health of an individual, such as a non-human mammal. For example, administration of a composition that includes R-beta-hydroxybutyrate (e.g., and/or other compounds) may improve and/or maintain an individual's health.

In some implementations, the administration of R-beta-hydroxybutyrate may be supplemented with other forms of beta-hydroxybutyrate, butyric acid, and/or butyrate.

In some implementations, the composition administered may include R-beta-hydroxybutyrate. The amount of R-beta-hydroxybutyrate included in the composition may be selected to obtain a result (e.g., induce ketosis; maintain ketosis; increase ketone levels, mental acuity, strength, etc.) upon administration (e.g., a pharmaceutically effective amount may be administered at a dosage and/or over a predetermined time period). In some implementations, the dosage and/or frequency of dosage may vary over time (e.g., initial vs. a lower dosage for maintenance, vary based on time of day, vary based on whether taken with or without a meal, etc.).

The R-beta-hydroxybutyrate in the composition may include any appropriate number of forms, such as salts, derivatives (e.g., esters), polymers, and/or complexes with other compounds. For example, the composition may include R-beta-hydroxybutyrate salt (e.g., sodium R-beta-hydroxybutyrate, magnesium R-beta-hydroxybutyrate, and/or potassium R-beta-hydroxybutyrate) and/or another form of R-beta-hydroxybutyrate (e.g., ester, polymer, complex, etc.). In some implementations, the composition may include an ester of R-beta-hydroxybutyrate. The composition may include an amino acid (e.g., separate and/or complexed with R-beta-hydroxybutyrate), such as leucine. The use of non-salt base R-beta-hydroxybutyrate may increase animal satisfaction (e.g., by reducing the cation, such as sodium and/or potassium load due to ingestion of the composition, by decreasing side effects, etc.), increase the applicability of the administration (e.g., since users sensitive to the cations of the R-beta-hydroxybutyrate salts may be less sensitive to the non-salt and/or lower salt plus non-salt forms of the composition). The administration of the composition may increase blood ketone levels, induce ketosis, maintain blood ketone levels, maintain ketosis, increase health, increase strength, increase mental acuity, etc. In some implementations, a first composition that includes R-beta-hydroxybutyrate salt may be administered to cause a first impact (e.g., induce ketosis, quickly increase mental acuity, quickly increase strength, etc.) and a second composition that includes non-salts R-beta-hydroxybutyrate (e.g., esters, polymers, complexes, etc.) and/or lower levels of R-beta-hydroxybutyrate salt may be utilized to cause a second impact (e.g., maintain ketosis, maintain mental acuity, maintain increased strength, etc.).

In some implementations, the form(s) of R-beta-hydroxybutyrate included may be selected based on the delivery form. For example, in some forms of food products the composition may include R-beta-hydroxybutyrate polymer (e.g., due to taste since increased cations like sodium may decrease palatability; due to nutrition since increased cations such as sodium may decrease nutrition; due to mixability, etc.). As another example, the composition may include R-beta-hydroxybutyrate salts or other forms (e.g., microencapsulated) to provide quick dissolve powders.

In various implementations, a composition may include R-beta-hydroxybutyrate and one or more additional compound(s). The R-beta-hydroxybutyrate may be in any appropriate form (e.g., salt, ester, polymer, complex, derivatives thereof, and/or combinations thereof). The additional compound(s) may be capable of independently increasing blood ketone levels (e.g., fatty acids or esters, berberine or berberine metabolites such as dihydroberberine, etc.). Additional compounds may include amino acids and/or proteins. Additional compound(s) may be capable of independently decreasing blood glucose levels (e.g., berberine or berberine metabolites such as dihydroberberine). In some implementations, additional compounds may not be capable of independently increasing blood ketone levels and/or decreasing blood glucose levels (e.g., additives, flavorings, colorings, minerals, vitamins, binders, anti-caking agents, etc.). The composition may be administered in an effective amount to cause a predetermined health impact (e.g., predetermined level of ketosis, blood ketone level, mental acuity, strength increase, perceived energy, fat loss, weight loss, etc.).

The composition may be administered to an individual in a predetermined amount and/or different amounts over an administration schedule. In some implementations, once a first criteria is satisfied (e.g., period of time, number of doses, predetermined health impact), the dosage amount may be altered. For example, a first dose(s) of the composition may be administered to cause a predetermined health impact and an additional lower dose(s) of the composition may be administered to maintain the predetermined health impact (e.g., caused in part by the first doses).

The composition may be administered in any appropriate delivery form (e.g., solid tablet; capsule; food products such as powdered products that can be mixed into food, mixed into beverages, and/or consumed directly; beverage product; etc.). The composition may be administered according to any appropriate schedule (e.g., periodic dosages, dosages as user desires, etc.). The administration schedule may inhibit administration that elevates blood ketone levels too high, decreases blood glucose levels too low, and/or causes an animal to consume a dosage that substantially elevates the risk of adverse and/or side effects, in some implementations.

In some implementations, the composition may include a long-acting component and/or be long-acting. For example, since animals have a high metabolism and process beta-hydroxybutyrate quickly, polymers and/or esters of beta-hydroxybutyrate (e.g., R-beta-hydroxybutyrate) may be included in the composition. The delivery of R-beta-hydroxybutyrate may be slower than the digestion of a beta-hydroxybutyrate salt (e.g., R-beta-hydroxybutyrate salt). In some implementations, the composition may include a R-beta-hydroxybutyrate and a long-acting R-beta-hydroxybutyrate form (e.g., polymer, ester, coated and/or processed form to provide slow release). In some implementations, a first dose(s) may include at least one non-long-acting form of beta-hydroxybutyrate and a second dose(s) may include at least one long-acting form of beta-hydroxybutyrate. The first dose(s) may be administered to cause a predetermined health impact and the second dose(s) may be administered to maintain the predetermined health impact. In some implementations, users may select the appropriate dose based on user preference and/or properties (e.g., a user on a ketogenic diet may chose the second dose since the user may already be in ketosis).

In some implementations, one or more additives may be included in the composition, such as flavorings (e.g., natural and/or artificial), vitamins, minerals, binders, and/or any other appropriate additive. The additives may alter flavor, color, and/or texture. The additives may increase palatability and/or facilitate inclusion in a delivery vehicle (e.g., tablet, food product, beverage product such as a drink mix, etc.). With non-human animals, compliance with administration schedules may be facilitated by increasing palatability and/or texture. The additive may be any appropriate solid and/or liquid to which the compound is added. For example, an additive may include liquid carriers, such as water and/or any other appropriate drinkable liquid. In some implementations, the composition may include a pharmaceutically inert liquid carrier, such as water (e.g., tap water, filtered water, distilled water, etc.). The liquid carrier may include other drinkable liquids. The liquid carrier may include an electrolyte solution, in some implementations.

The described compositions may be administered via any appropriate administration method. For example, the described compositions may be administered enterally and/or parenterally. In some implementations, the described composition may be administered via a tablet and/or capsule. The described composition may be provided in a powdered form that allows the described composition to be sprinkled on food, mixed with a liquid to provide a beverage, and/or directly administered. The described composition may be provided in gel form and/or a bolus. The compounds in the composition may be mixed, coupled to each other, and/or provided separately. For example, the composition may include beta-hydroxybutyrate coupled to another compound (e.g., beta-hydroxybutyrate ester and/or amino acid). In some implementations, the beta-hydroxybutyrate and one or more other compounds may be provided separately (e.g., in pills). A non-human animal may sequentially and/or concurrently be administered (e.g., swallow pills) the beta-hydroxybutyrate and other compounds.

The described compositions may be administered on an administration protocol to cause predetermined effects on non-human animals. For example, the described compositions may be administered once a day, via an extended release preparation, and/or multiple times a day (e.g., 1 to 5 times a day, 2 to 5 times a day, 3 to 5 times a day, etc.). The described composition may replace other pharmaceuticals or dietary supplements taken and/or be utilized in combination with one or more other pharmaceuticals or dietary supplements, as appropriate. The described composition may replace other pharmaceuticals or dietary supplements taken and/or be utilized in combination with one or more other pharmaceuticals or dietary supplements, as appropriate, in some implementations.

In various implementations, the described composition(s) (e.g., butyrate, beta-hydroxybutyrate, R-beta-hydroxybutyrate, related compounds, and/or one or more other compounds) may include one or more of the described components, equivalent(s) of the described component(s), derivatives of the described component(s), complex(es) of the described component(s), salt(s) of the described component(s), and/or combinations thereof.

In various implementations, an effective amount of one or more of the described composition(s) may be administered. Administration of the effective amount may induce and/or maintaining ketosis; maintaining and/or promoting weight loss; increase mental processes (e.g., acuity including cognitive functioning, mood, energy, alertness, focus, performance, effects of aging, etc.); improve and/or maintain body composition; function as a therapeutic for one or more of the described conditions or disorders (e.g., treat neurological disorders); and/or combinations thereof.

Although various types of increases in mental acuity have been described, other features of mental acuity such as memory, focus, concentration, and/or understanding (e.g., speed of processing, accuracy of processing) may be increased by administration of an effective amount of the composition that includes R-beta-hydroxybutyrate.

Although a subject and/or an individual have been described as a non-human mammal or animal, a subject and/or individual may be a non-human mammal or a group of non-human mammals. The animal may be non-human mammals, such as cats, dogs, cattle (e.g., cows and bulls), horses, sheep, pigs, etc.

In various implementations, beta-hydroxybutyrate may administered simultaneously and/or sequentially with one or more other compounds (e.g., short chain, medium chain, and/or long chain fatty acids). For example, beta-hydroxybutyrate and/or one or more other compounds may be delivered mixed in a powdered, liquid, gel, and/or other appropriate form. In some implementations, the beta-hydroxybutyrate and/or one or more other compounds may be administered via pills, tablets, capsules, other oral administration forms, intravenously, nasal sprays, sublingual tabs/strips, or topical delivery, rectal, other appropriate administration forms, and/or combinations thereof.

Although the term beta-hydroxybutyrate is the terminology used in the described implementations, beta-hydroxybutyrate is also referred to as beta-hydroxybutyrate, (R)-3-Hydroxybutyric acid, (R)-3-Hydroxybutanoic acid, (3R)-3-hydroxybutanoic acid, (R)-3-Hydroxybutanoate, (R)-(-)-3-Hydroxybutyric acid, (R)-(-)-beta-Hydroxybutyric acid, 3-D-hydroxybutyrate, BHIB, BHB, 3-delta-hydroxybutyrate, delta-3-hydroxybutyrate, 3-D-hydroxybutyric acid, D-3-hydroxybutyric acid, 3R-hydroxy-butanoic acid, delta-beta-hydroxybutyrate, D-3-hydroxybutyrate, D-(-)-3-hydroxybutyrate, delta-3-hydroxybutyric acid, (-)-3-Hydroxybutyric acid, D-beta-hydroxybutyrate, (R)-(-)-b-Hydroxybutyrate, (R)-beta-Hydroxybutyric acid, delta-(-)-3-hydroxybutyrate, (R)-3-hydroxybutyrate, (R)-beta-Hydroxybutanoic acid, (R)-(-)-beta-hydroxybutyrate, (-)-3-Hydroxy-n-butyric acid, (R)-(-)-b-Hydroxybutyric acid, Butanoic acid, 3-hydroxy-, (R)-Butyric acid, 3-hydroxy-, D-(-)-(R)-3 -82578-46-9, beta-D-Hydroxybutyric acid, D-beta-Hydroxybutyric acid, (3R)-3-delta-hydroxybutyric acid, 3-(R)-Hydroxybutyric acid, and/or (-)-beta-Hydroxybutyrate.

In various implementations, beta-hydroxybutyrate is a ketone body component and included in a ketone body composition; administered in an amount, form, and/or schedule; and/or being in a particular form (e.g., complexed and/or coupled). R-beta-hydroxybutyrate may be utilized in the various described implementations of beta-hydroxybutyrate in the same or lower amount as the described beta-hydroxybutyrate, as appropriate.

It is to be understood the implementations are not limited to particular systems or processes described which may, of course, vary. It is also to be understood that the terminology used herein is for the purpose of describing particular implementations only and is not intended to be limiting. As used in this specification, the singular forms “a”, “an” and “the” include plural referents unless the content clearly indicates otherwise. Thus, for example, reference to “a compound” includes a combination of two or more compounds and reference to “beta-hydroxybutyrate” includes different types and/or combinations of beta-hydroxybutyrate.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method for raising blood ketone levels in a non-human mammal in order to therapeutically or prophylactically treat one or more conditions selected from obesity, muscle atrophy, body composition, gut health, disease, cancer, epilepsy, seizures, Alzheimer's, oxidative stress, vascular disease, disrupted mitochondria, metabolic decline, cognitive decline, brain function, brain metabolism, brain atrophy, mental acuity, neurological disorders, inflammation, joint health, motor control, memory, or mood, the method comprising:
orally administering an effective amount of a ketone body composition to a non-human mammal in need thereof to raise blood ketone levels in the non-human animal and therapeutically or prophylactically treat the one or more conditions,
the ketone body composition comprising a ketone body component or precursor selected from the group consisting of beta-hydroxybutyrate salts, beta-hydroxybutyrate esters, beta-hydroxybutyrate acid, beta-hydroxybutyrate oligomers or polymers, acetoacetate salts, acetoacetate esters, acetoacetic acid, 1,3-butanediol, and combinations thereof,
wherein the ketone body composition is in a form conducive for oral intake by the non-human mammal through one or more of a flavored liquid carrier, a flavored gel, an electrolyte solution, a broth, or a coating material for animal food.

2. The method of claim 1, wherein the non-human animal is selected from a dog, cat, horse, cattle, sheep, or pig.

3. The method of claim 1, wherein administering the effective amount of the ketone body composition provides a daily dose of about 0.5 grams to about 200 grams, or about 50 g to 100 g, or about 1 g to 15 g of beta-hydroxybutyrate in either a single dose or in multiple doses.

4. The method of claim 1, the ketone body composition further comprising an amino acid selected from the group consisting of leucine, lysine, arginine, histidine, ornithine, creatine, agmatine, citrulline, and combinations thereof.

5. The method of claim 1, further comprising administering an effective amount of at least one of butyrate, butyric acid, or tributyrin to the non-human mammal to improve gut health.

6. The method of claim 5, wherein the amount of butyrate and/or butyric acid administered is greater than the amount of the ketone body component administered.

7. The method of claim 5, wherein the amount of butyrate and/or butyric acid administered is approximately 0.1 g to approximately 20 g butyrate and/or butyric acid in either a single dose or in multiple doses.

8. The method of claim 1, wherein the ketone body component is encapsulated, incorporated into a microemulsion or into liposomes, agglomerated, processed using masking/flavoring technologies, and/or otherwise processed so as to reduce organoleptic reactions from the non-human mammal.

9. The method of claim 1, wherein the ketone body component is administered as bone broth, a powder, gel or liquid added to animal food, an animal treat, or an animal drink.

10. The method of claim 1, the ketone body composition further comprising one or more of medium chain fatty acids having 6 to 12 carbons, medium chain triglycerides, long-chain fatty acids having more than 12 carbons, or long-chain triglycerides.

11. The method of claim 1, further comprising administering an effective amount of one or more of berberine, dihydroberberine, or tetrahydroberberine for to reduce blood glucose level in the non-human mammal.

12. The method of claim 1, wherein administering the ketone body composition slows or reverses cognitive decline in the non-human mammal.

13. The method of claim 1, wherein administering the ketone body composition reduces inflammation in the non-human mammal.

14. The method of claim 1, wherein administering the ketone body composition reduces or slows the growth of cancerous tumors in the non-human mammal.

15. The method of claim 1, wherein administering the ketone body composition provides at least one of weight management, improved body composition, or increased lean to fat ratio in the non-human mammal.

16. The method of claim 1, wherein administering the ketone body composition supports barrier function(s) in the gut and/or reduces inflammation associated with ulcerative colitis in the non-human mammal.

17. A ketone body composition for use in raising blood ketone levels in a non-human mammal in order to therapeutically or prophylactically treat one or more conditions selected from obesity, muscle atrophy, body composition, gut health, disease, cancer, epilepsy, seizures, Alzheimer's, oxidative stress, vascular disease, disrupted mitochondria, metabolic decline, cognitive decline, brain function, brain metabolism, brain atrophy, mental acuity, neurological disorders, inflammation, joint health, motor control, memory, or mood, the ketone body comprising:
at least one of animal food, animal drink, or composition formulated for addition to animal food or animal drink; and
an effective amount of a ketone body composition to a non-human mammal in need thereof to raise blood ketone levels in the non-human animal and therapeutically or prophylactically treat the one or more conditions, the ketone body composition comprising a ketone body component or precursor selected from the group consisting of beta-hydroxybutyrate salts, beta-hydroxybutyrate esters, beta-hydroxybutyrate acid, beta-hydroxybutyrate oligomers or polymers, acetoacetate salts, acetoacetate esters, acetoacetic acid, 1,3-butanediol, and combinations thereof,
wherein the ketone body component is encapsulated, incorporated into a microemulsion or into liposomes, agglomerated, processed using masking/flavoring technologies, and/or otherwise processed so as to reduce organoleptic reactions from the non-human mammal.

18. The ketone body composition of claim 17, wherein the non-human animal is selected from a dog, cat, horse, cattle, sheep, or pig.

19. The ketone body composition of claim 17, wherein the ketone body composition is in a dosage form that provides a daily dose of about 0.5 grams to about 200 grams, or about 50 g to 100 g, or about 1 g to 15 g of beta-hydroxybutyrate in either a single dose or in multiple doses.

20. The method of claim 17, further comprising an effective amount of butyrate, butyric acid, or tributyrin for improving gut health in the non-human mammal.
